(19) United States
(12) Patent Application Publication
(10) Pub. No.: US 2006/0110350 A1
(43) Pub. Date: May 25, 2006

(54) Composition comprising electrophilic monomers and particular organic salts, and use thereof for cosmetic treatment of keratin materials

(76) Inventors: Henri Samain, Bievres (FR); Franck Giroud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,585

(22) Filed: Oct. 13, 2005

Related U.S. Application Data
(60) Provisional application No. 60/646,500, filed on Jan. 25, 2005.

(30) Foreign Application Priority Data
Oct. 13, 2004 (FR) 04 10810

(51) Int. Cl. A61K 8/81

(52) U.S. Cl. 424/70.11

(57) ABSTRACT

The present invention relates to a composition comprising in a cosmetically acceptable medium at least one electrophilic monomer and at least one non-polymeric organic salt having a melting point of less than 60° C., and to its use for the cosmetic treatment of keratin materials. It also relates to a method of cosmetic treatment of keratin materials, especially keratin fibres such as the hair, which employs said composition. It likewise relates to a method of cosmetic treatment of keratin materials which comprises one step of applying said non-polymeric organic salt and another step of applying at least one electrophilic monomer.

The present invention relates to a composition comprising at least one electrophilic monomer and at least one particular organic salt, to its use for the cosmetic treatment of keratin materials, and to a cosmetic treatment method employing such a composition.

Within the field of cosmetology attempts are made to modify the surface properties of keratin materials, such as keratin fibres, the hair for instance, in order to provide the hair, for example, with a conditioning effect, such as softness, or sheen. This is generally done using cosmetic compositions based on conditioning agents such as silicones or polymers which have a high affinity for keratin materials, and especially for the hair.

However, these conditioning agents tend to be lost in the course of washing with shampoos, so making it necessary to repeat application of the compositions to the hair.

In order to increase the staying power of polymer deposits, it is possible to consider carrying out a free-radical polymerization of certain monomers directly on the hair. However, severe degradation of the hair fibres is observed, probably associated with the polymerization initiators, and the hair thus treated is difficult to disentangle.

The Applicant has found, surprisingly, that by using the combination of at least one particular non-polymeric organic salt with at least one electrophilic monomer as described below it is possible to obtain durably improved conditioning and sheen of the hair.

This is because a composition comprising such a combination makes it possible to maintain the softness and sheen provided to the hair by said composition, without repeat application, even after the hair has been washed a number of times.

Applying a composition comprising such a combination leads to the formation in situ of a glossy, lubricating coating which has staying power, particularly in the face of shampooing.

Moreover, the individual hairs, surprisingly, remain completely separate and can be styled without problem.

The invention accordingly provides a composition comprising in a cosmetically acceptable medium at least one electrophilic monomer and at least one non-polymeric organic salt having a melting point of less than 60° C.

Another subject of the present invention consists in the use of said composition for the cosmetic treatment of keratin materials, and more particularly of keratin fibres such as the hair.

The invention further provides a method of cosmetic treatment of keratin materials, and more particularly of keratin fibres such as the hair, which employs said composition.

Other subjects, features, aspects and advantages of the invention will appear more clearly still when the description and examples which follow are read.

According to the invention the composition comprises in a cosmetically acceptable medium at least one electrophilic monomer and at least one non-polymeric organic salt having a melting point of less than 60° C.

The organic salts which can be used in the present invention form part of a general class of compounds which are well known under the name “room temperature ionic liquids or RTIL”. These RTIL generally exhibit a melting point of less than 100° C. and remain liquid up to a temperature of approximately 300° C.

These RTIL are described in particular in “Eyes on Ionic Liquids, Chemical and Engineering News”, May 15, 2000, Vol. 78, 20, pages 37-50, and in “New Horizons For Ionic Liquids, Chemical and Engineering News”, Jan. 1, 2001, Vol. 79, 1, pages 21-25.

The melting point is measured by differential calorimetric analysis (DSC) with a rate of temperature rise of 10° C./min. The melting point is then the temperature corresponding to the summit of the endothermic melting peak obtained in the course of measurement.

The melting point of the organic salt is preferably less than 20° C., more preferably less than 0° C. and more preferably still less than −30° C.

The organic salts used in the context of the invention additionally possess an excellent solvency and an excellent electrical conductivity. They are also non-volatile and non-flammable.

Another advantage of these organic salts resides in the fact that they are readily recyclable and form part of solvents known as “green” solvents.

When combined with at least one electrophilic monomer as described below, they allow a glossy, lubricating coating to be obtained which exhibits staying power.

Preferably the organic salts may be selected from imidazolium salts, pyrazolium salts, pyridinium salts, pyrimidinium salts, tetraalkylphosphonium salts and tetraalkylammonium salts, with a melting point of less than 60° C., and mixtures thereof.

Examples of imidazolium salts include in particular those of formula (I): 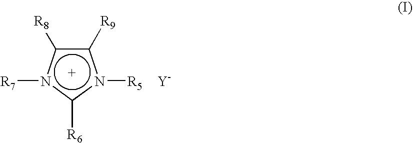
in which:
R₅ and R₇, which are identical or different, each represent an alkyl group containing 1 to 12 carbon atoms, preferably 1 to 5 carbon atoms and more preferably 1 to 4 carbon atoms,
R₆, R₈ and R₉, which are identical or different, each represent a hydrogen atom or an alkyl group containing 1 to 4 carbon atoms and preferably 1 to 3 carbon atoms, and
Y⁻ represents an anion.

The alkyl groups may be linear or branched. Mention may be made in particular, for example, of methyl, ethyl, n-propyl, isopropyl, n-butyl, tert-butyl, pentyl, hexyl, octyl, nonyl, decyl and dodecyl groups.

In the composition of the invention it is preferred to use (C₁₋₁₀ alkyl)-methylimidazolium salts, and more specifically 1-butyl-3-methylimidazolium or 1-ethyl-3-methylimidazolium salts.

Pyrazolium salts which can be used in the present invention include in particular those of formula (II): 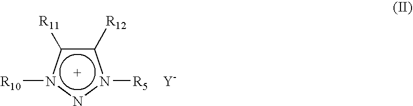
in which:
R₅ and Y⁻ have the same signification as above and
R₁₀, R₁₁, and R₁₂, which are identical or different, each represent a hydrogen atom or an alkyl group containing 1 to 12 carbon atoms and preferably 1 to 4 carbon atoms.

Examples of pyridinium salts which can be used in the composition of the invention include in particular those of formula (III): 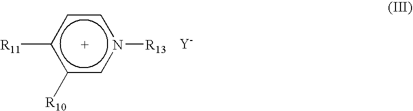
in which R₁₀, R₁₁, and Y⁻ are as defined above and R₁₃ represents an alkyl group containing 1 to 12 carbon atoms and preferably 1 to 4 carbon atoms;
and more particularly N-butylpyridinium salts.

Pyrimidinium salts which can be used in the present invention include in particular those of formulae (IV) and (IV′): 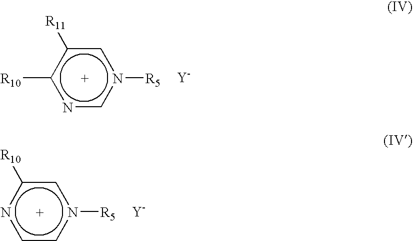
in which R₅, R₁₀, R₁₁, and Y⁻ are as defined above.

Other organic salts exist in the form of non-heterocyclic cations, such as tetraalkylphosphonium salts and tetraalkylammonium salts with a melting point of less than 60° C.

Tetraalkylphosphonium or tetraalkylammonium salts which can be used in the present invention include in particular those of formula (V) or (VI): 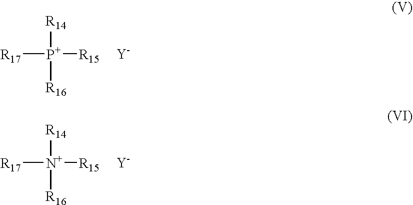
in which R₁₄, R₁₅, R₁₆ and R₁₇, which are identical or different, represent an alkyl group containing 1 to 18 carbon atoms and preferably 1 to 14 carbon atoms, and Y⁻ represents an anion.

The anion present in the organic salt, represented by Y⁻, may be any anion which is well known in the art, and in particular a chloride ion (Cl⁻), a bromide ion (Br⁻), a tetrachloroaluminate ion (AlCl₄⁻), a tetrachloronickel ion (NiCl₄⁻), a perchlorate ion (ClO₄⁻), a nitrate ion (NO₃⁻), a nitrite ion (NO₂⁻), a sulphate ion (SO₄²⁻), a methyl sulphate ion (CH₃SO₄⁻), a tetrafluoroborate ion (BF₄⁻), a hexafluorophosphate ion (PF₆⁻), a hexafluoroantimonate ion (SbF₆⁻), a triflate ion [TfO] (CF₃SO₂⁻), a nonaflate ion [NfO] (CF₃(CF₂)₃SO₂⁻), an ion [Tf₂N] (CF₃SO₂)₂N⁻), a trifluoroacetate ion [TA] (CF₃CO₂⁻), a heptafluorobutanoate ion [HB] (CF₃(CF₂)₃CO₂⁻), an acetate ion (CH₃CO₂⁻), or a trifluoromethanesulphonate ion (CF₃SO₃⁻).

Among the abovementioned anions very particular preference is given to chloride, bromide, sulphate, acetate, tetrafluoroborate, hexafluorophosphate, triflate, nonaflate, [Tf₂N] (CF₃SO₂)₂N⁻) and heptafluorobutanoate ions.

Other classes of organic salts which can be used in the compositions of the invention are cited in application WO 2004/035018.

As examples of organic salts which can be used in the context of the present invention, mention may be made in particular of the following organic salts:
1-ethyl-3-methylimidazolium chloride,
1-ethyl-3-methylimidazolium bromide,
1-butyl-3-methylimidazolium chloride,
1-hexyl-3-methylimidazolium chloride,
1-methyl-3-octylimidazolium chloride,
1-decyl-3-methylimidazolium chloride,
1-decyl-3-methylimidazolium bromide,
1-dodecyl-3-methylimidazolium chloride,
1-methyl-3-tetradecylimidazolium chloride,
4-methyl-N-butyl-pyridinium chloride,
3-methyl-N-butylpyridinium chloride,
4-methyl-N-hexylpyridinium chloride,
1-ethyl-3-methylimidazolium tetrafluoroborate,
1-butyl-3-methylimidazolium tetrafluoroborate,
1-pentyl-3-methylimidazolium tetrafluoroborate,
1-hexyl-3-methylimidazolium tetrafluoroborate,
1-heptyl-3-methylimidazolium tetrafluoroborate,
1-octyl-3-methylimidazolium tetrafluoroborate,
1-nonyl-3-methylimidazolium tetrafluoroborate,
1-decyl-3-methylimidazolium tetrafluoroborate,
4-methyl-N-butylpyridinium tetrafluoroborate,
1-hexyl-3-ethylimidazolium tetrafluoroborate,
1-ethyl-3-methylimidazolium hexafluorophosphate,
1-butyl-3-methylimidazolium hexafluorophosphate,
1-pentyl-3-methylimidazolium hexafluorophosphate,
1-hexyl-3-methylimidazolium hexafluorophosphate,
1-heptyl-3-methylimidazolium hexafluorophosphate,
1-octyl-3-methylimidazolium hexafluorophosphate,
1-nonyl-3-methylimidazolium hexafluorophosphate,
1-decyl-3-methylimidazolium hexafluorophosphate,
1,3-dimethylimidazolium methyl sulphate,
1-methyl-3-butylimidazolium methyl sulphate,
1-ethyl-3-methylimidazolium nitrate,
1-ethyl-3-methylimidazolium nitrite,
1-ethyl-3-methylimidazolium acetate,
1-ethyl-3-methylimidazolium sulphate,
1-ethyl-3-methylimidazolium triflates,
1-ethyl-3-methylimidazolium nonaflates,
1-ethyl-3-methylimidazolium bis(trifyl)amide,
1-butylpyridinium bromide,
1-butylpyrimidinium trifluoromethanesulphonate,
1-hexylpyrimidinium trifluoromethanesulphonate,
1-ethyl-3-methylimidazolium trifluoroacetate,
trihexyltetradecylphosphonium chloride,
tributyltetradecylphosphonium chloride,
1-ethyl-2-methylpyrazolium tetrafluoroborate,
1-methyl-3-butylpyrimidinium tetrafluoroborate, and
trioctylammonium sulphate.

It is possible to modify the chemistry of the organic salts, such as it has been described above, in order to vary their solubility. In order to preserve the low-temperature meltability of these salts it is preferable to modify, more specifically, the alkyl chains partly making up the organic salts. In this context mention may be made, by way of example, of the etherification of the alkyl chain of salts of the 1-alkyl-3-methylimidazolium class, which makes it possible to obtain the compounds of formulae:

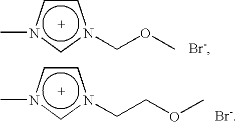

Chemical modification of this kind imparts or reinforces, for the organic salt, the water-solubility, without loss of the low-temperature meltability of the salt. Chemical modification of this kind may also make it possible to cause the organic salts to gel by combination with amphiphilic sugars and to give what are called ionogels. Such properties are described in the article “Spontaneous Self assembly of glycolipid bilayer Membranes in Sugar-philic Ionic Liquids and formation of Ionogels”, published by N. Kimizuka and T Nakashima (Langmuir 17, 6759-61 (2001)).

The organic salt or the mixture of organic salts as described above is present in the cosmetic compositions of the invention at a concentration of between 0.001% and 95% by weight, preferably between 0.1% and 50% by weight, and more preferably between 0.2% and 20% by weight, relative to the total weight of the composition.

The organic salt or mixture of organic salts may be present as it is or in solution or in the form of an emulsion. The organic salt or the mixture of organic salts may also be microencapsulated beforehand, before incorporation into the cosmetic composition.

An electrophilic monomer is a monomer capable of undergoing polymerization by anionic polymerization in the presence of a nucleophile such as, for example, the hydroxyl (OH⁻) ions present in water.

By anionic polymerization is meant the mechanism defined in the work “Advanced Organic Chemistry”, Third Edition, Jerry March, pages 151 to 161.

The one or more electrophilic monomers present in the composition of the invention may be selected from:
(i) benzylidenemalononitrile derivatives (A), 2-(4-chlorobenzylidene)malononitrile (A1), ethyl 2-cyano-3-phenylacrylate (B) and ethyl 2-cyano-3-(4-chlorophenyl) acrylate (B1), as are described in Sayyah, J. Polymer Research, 2000, p. 97: 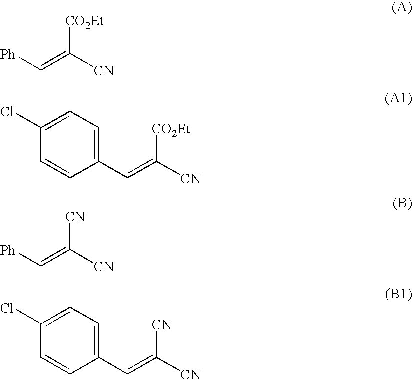
(ii) methylidenemalonate derivatives such as:
   diethyl 2-methylenemalonate (C) as described by Hopff, Makromolekulare Chemie, 1961, p. 95, by De Keyser, J. Pharm. Sci, 1991, p. 67, and by Klemarczyk, Polymer, 1998, p. 173: 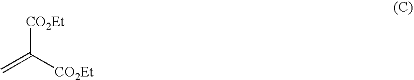
   ethyl 2-ethoxycarbonylmethyleneoxycarbonylacrylate (D), as described by Breton, Biomaterials, 1998, p. 271 and Couvreur, Pharmaceutical Research, 1994, p. 1270: 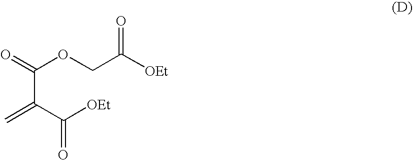
(iii) itaconate and itaconimide derivatives such as:
   dimethyl itaconate (E), as described by Bachrach, European Polymer Journal, 1976, p. 563: 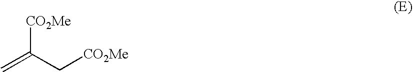
   N-butylitaconimide (F), N-(4-tolyl)itaconimide (G), N-(2-ethylphenyl)itaconimide (H) and N-(2,6-diethylphenyl)itaconimide (I), as are described by Wanatabe, J. Polymer Science: Part A: Polymer Chemistry, 1994, p. 2073: 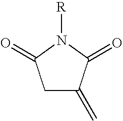
      R=Bu (F), 4-tolyl (G), 2-ethylphenyl (H), 2,6-diethylphenyl (I)
(iv) methyl α-(methylsulphonyl)acrylate derivatives (K), ethyl α-(methylsulphonyl)acrylate derivatives (L), methyl α-(tert-butylsulphonyl)acrylate derivatives (M), tert-butyl α-(methylsulphonyl)acrylate derivatives (N) and tert-butyl α-(tert-butylsulphonyl)acrylate derivatives (O), as are described by Gipstein, J. Org. Chem, 1980, p. 1486, and 1,1-bis(methylsulphonyl)ethylene derivatives (P), 1-acetyl-1-methylsulphonylethylene derivatives (Q), methyl α-(methylsulphonyl)vinylsulphonate derivatives (R) and α-methylsulphonylacrylonitrile derivatives (S), as are described by Shearer, U.S. Pat. No. 2,748,050: 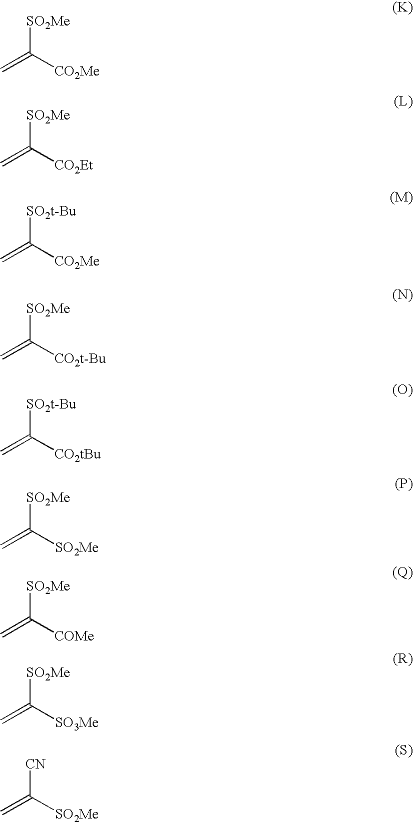
(v) methyl vinyl sulphone derivatives (T) and phenyl vinyl sulphone derivatives (U), as are described by Boor, J. Polymer Science, 1971, p. 249: 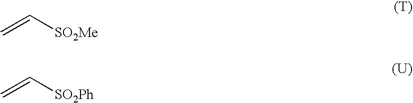
(vi) the phenyl vinyl sulphoxide derivative (V), as described by Kanga, Polymer Preprints (ACS, Division of Polymer Chemistry), 1987, p. 322: 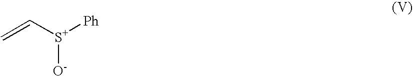
(vii) the 3-methyl-N-(phenylsulphonyl)-1-aza-1,3-butadiene derivative (W), as described by Bonner, Polymer Bulletin, 1992, p. 517: 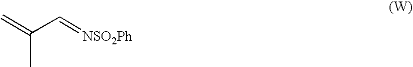
(viii) acrylate and acrylamide derivatives such as:
   N-propyl-N-(3-triisopropoxysilylpropyl)acrylamide (X) and N-propyl-N-(3-triethoxysilylpropyl)acrylamide (Y), as described by Kobayashi, Journal of Polymer Science, Part A: Polymer Chemistry, 2005, p. 2754: 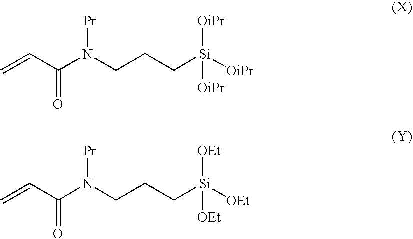
   2-hydroxyethyl acrylate (Z) and 2-hydroxyethyl methacrylate (AA), as are described by Rozenberg, International Journal of Plastics Technology, 2003, p. 17: 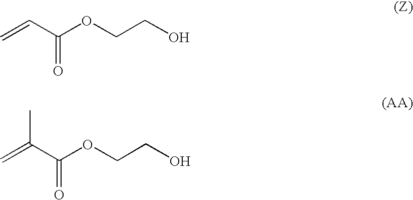
   n-butyl acrylate (AB), as described by Schmitt, Macromolecules, 2001, p. 2115, and tert-butyl acrylate (AC), as described by Ishizone, Macromolecules, 1999, p. 955:

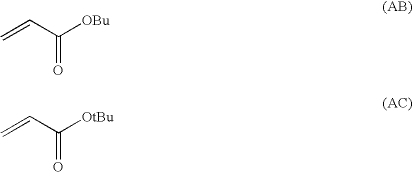

The electrophilic, or electron-withdrawing, monomer useful in the present invention may be cyclic or linear. When it is cyclic, the electron-withdrawing group is preferably exocyclic, which is to say that it does not form an integral part of the cyclic structure of the monomer.

According to one particular embodiment these monomers have at least two electron-withdrawing groups.

Examples of electrophilic monomers having at least two electron-withdrawing groups include the monomers of formula (1): 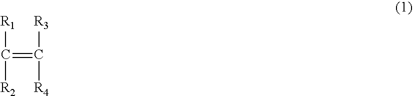
in which:
R₁ and R₂ each denote, independently of one another, a minimally or non-electron-withdrawing group (with little or no inductive withdrawal effect), such as:
   a hydrogen atom,
   a saturated or unsaturated linear, branched or cyclic hydrocarbon group, containing preferably 1 to 20, more preferably 1 to 10, carbon atoms, and optionally containing one or more nitrogen, oxygen and/or sulphur atoms, which is optionally substituted by one or more groups selected from —OR, —COOR, —COR, —SH, —SR, —OH and halogen atoms,
   a modified or non-modified polyorganosiloxane residue, and
   a polyoxyalkylene group,
R₃ and R₄ each denote, independently of one another, an electron-withdrawing (or inductively withdrawing) group selected preferably from the groups —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH, linear or branched alkenyl groups, linear or branched alkynyl groups, C₁-C₄ mono- or polyfluoroalkyl groups, aryl groups such as phenyl, or aryloxy groups such as phenyloxy,
R denotes a saturated or unsaturated linear, branched or cyclic hydrocarbon group containing preferably 1 to 20, more preferably 1 to 10, carbon atoms, and optionally containing one or more nitrogen, oxygen and/or sulphur atoms, which is optionally substituted by one or more groups selected from —OR′, —COOR′, —COR′, —SH, —SR′, —OH, halogen atoms and a polymer residue obtainable by free-radical polymerization or by polycondensation or by ring opening, R′ denoting a C₁-C₁₀ alkyl group.

By electron-withdrawing or inductively withdrawing group (—I) is meant any group which is more electronegative than carbon. Reference may be made to the work by P. R. Wells in Prog. Phys. Org. Chem., Vol. 6, 111 (1968).

A minimally or non-electron-withdrawing group is any group whose electronegativity is less than or equal to that of carbon.

The alkenyl or alkynyl groups have preferably 2 to 20 carbon atoms, more preferably 2 to 10 carbon atoms.

As a saturated or unsaturated linear, branched or cyclic hydrocarbon group, containing preferably 1 to 20 carbon atoms, more preferably 1 to 10 carbon atoms, mention may be made in particular of linear or branched alkyl, alkenyl or alkynyl groups, such as methyl, ethyl, n-butyl, tert-butyl, isobutyl, pentyl, hexyl, octyl, butenyl or butynyl; and cycloalkyl or aromatic groups.

As a substituted hydrocarbon group mention may be made, for example, of hydroxyalkyl or polyhaloalkyl groups.

As examples of non-modified polyorganosiloxane mention may be made in particular of polyalkylsiloxanes such as polydimethylsiloxanes, polyarylsiloxanes such as polyphenylsiloxanes, and polyarylalkylsiloxanes such as polymethylphenylsiloxanes.

Among modified polyorganosiloxanes, mention may be made in particular of polydimethylsiloxanes containing polyoxyalkylene and/or siloxy and/or silanol and/or amine and/or imine and/or fluoroalkyl groups.

Among polyoxyalkylene groups mention may be made in particular of polyoxyethylene groups and polyoxypropylene groups having preferably 1 to 200 oxyalkylene units.

Among mono- or polyfluoroalkyl groups mention may be made in particular of groups such as —(CH₂)ₙ—(CF₂)ₘ—CF₃ or —(CH₂)ₙ—(CF₂)ₘ—CHF₂ with n=1 to 20 and m=1 to 20.

The substituents R₁ to R₄ may optionally be substituted by a group which has a cosmetic activity. The cosmetic activities used in particular are obtained from groups having colouring, antioxidant, UV filter and conditioning functions.

Examples of groups having a colouring function include, in particular, azo, quinone, methine, cyanomethine and triarylmethane groups.

Examples of groups having an antioxidant function include, in particular, groups of butylated hydroxyanisole (BHA) type, butylated hydroxytoluene (BHT) type or vitamin E type.

Examples of groups having a UV filter function include, in particular, groups of benzophenone, cinnamate, benzoate, benzylidenecamphor and dibenzoylmethane types.

Examples of groups having a conditioning function include, in particular, cationic groups and groups of fatty ester type.

Among the abovementioned monomers preference is given to the monomers from the class of the cyanoacrylates and their derivatives, of formula (2): 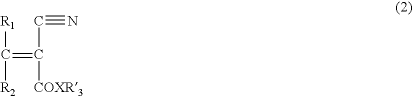
where
X denotes NH, S or O,
R₁ and R₂ have the same significations as above,
R′₃ can denote a hydrogen atom or a group R as defined for the formula (1).
Preferably X denotes O.

As compounds of formula (2), mention may be made of the monomers:
a) belonging to the class of C₁₋₂₀ perfluoroalkyl 2-cyanoacrylates, such as:
   the 2,2,3,3-tetrafluoropropyl ester of 2-cyano-2-propenoic acid, of formula: 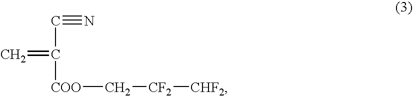
   or else the 2,2,2-trifluoroethyl ester of 2-cyano-2-propenoic acid, of formula: 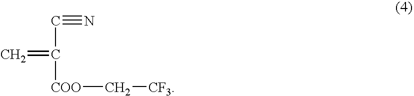
b) C₁-C₁₀ alkyl or (C₁-C₄)alkoxy(C₁-C₁₀)alkyl cyanoacrylates.

Mention may be made more particularly of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

In the context of the invention it is preferred to use monomers b).

The most particularly preferred monomers are those of formula (5) and mixtures thereof: 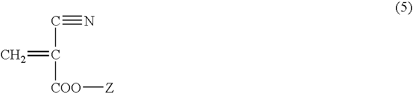
in which: Z=—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃,
—(CH₂)₄—CH(C₂H₅)—CH₃.

The monomers used in accordance with the invention may be attached covalently to supports such as polymers, oligomers or dendrimers. The polymer or oligomer may be of linear, branched, comb or block structure. The distribution of the monomers of the invention over the polymeric, oligomeric or dendritic structure may be random, terminal or blockwise.

According to the present invention, the monomers are preferably selected from monomers capable of undergoing polymerization on keratin fibres under cosmetically acceptable conditions. In particular the polymerization of the monomer takes place preferably at a temperature less than or equal to 80° C., preferably between 10 and 80° C., preferably 20 to 80° C., which does not prevent application being ended with an operation of drying under a hood, blow-drying or passage of a flat iron or curling tongs.

The compositions employed in accordance with the invention generally have a concentration of electrophilic monomer according to the invention of between 0.001% and 80% by weight, and more particularly between 0.1% and 40%, and more preferably still between 1% and 20% by weight relative to the total weight of the composition.

It is also possible to introduce polymerization inhibitors into the compositions, and more particularly free-radical and/or anionic polymerization inhibitors, in order to increase the stability of the composition over time. Without limitation, the following polymerization inhibitors may be mentioned: sulphur dioxide, nitric oxide, lactone, boron trifluoride, hydroquinone and its derivatives such as hydroquinone monoethyl ether, tert-butylhydroquinone (TBHQ), benzoquinone and its derivatives such as duroquinone, catechol and its derivatives such as tert-butylcatechol and methoxycatechol, anisole and its derivatives such as methoxyanisole, hydroxyanisole or butylated hydroxyanisole, pyrogallol, 2,4-dinitrophenol, 2,4,6-trihydroxybenzene, p-methoxyphenol, hydroxybutyltoluene, alkyl sulphates, alkyl sulphites, alkyl sulphones, alkyl sulphoxides, alkyl sulphides, mercaptans, 3-sulpholene, and mixtures thereof. The alkyl groups denote preferably groups having 1 to 6 carbon atoms.

It is also possible to use organic or inorganic acids, the organic acids having one or more carboxylic or sulphonic groups, which have a pKa of between 0 and 6, such as phosphoric acid, hydrochloric acid, nitric acid, benzene- or toluene-sulphonic acid, sulphuric acid, carbonic acid, hydrofluoric acid, acetic acid, formic acid, propionic acid, benzoic acid, mono-, di- or trichloroacetic acids, salicylic acid and trifluoroacetic acid.

The amount of inhibitor may range from 10 ppm to 20%, and more preferably from 10 ppm to 5%, and more preferably still from 10 ppm to 1% by weight relative to the total weight of the composition.

By cosmetically acceptable medium is meant a medium which is compatible with keratin materials such as the hair.

The cosmetically acceptable medium is preferably anhydrous. By an anhydrous medium is meant one containing less than 1% by weight of water relative to the total weight of the composition.

The cosmetically acceptable medium is preferably selected from organic oils; silicones such as volatile silicones, amino or non-amino silicone gums or oils and mixtures thereof; mineral oils; vegetable oils such as olive oil, castor oil, colza oil, copra oil, wheatgerm oil, sweet almond oil, avocado oil, macadamia oil, apricot oil, safflower oil, candlenut oil, false flax oil, tamanu oil and lemon oil; waxes; or else organic compounds such as C₅-C₁₀ alkanes, acetone, methyl ethyl ketone, esters of C₁-C₂₀ acids and C₁-C₈ alcohols such as methyl acetate, butyl acetate, ethyl acetate and isopropyl myristate, dimethoxyethane, diethoxyethane, C₁₀-C₃₀ fatty alcohols such as lauryl alcohol, cetyl alcohol, stearyl alcohol and behenyl alcohol, C₁₀-C₃₀ fatty acids such as lauric acid and stearic acid, C₁₀-C₃₀ fatty amides such as lauric diethanolamide, C₁₀-C₃₀ fatty alcohol esters such as C₁₀-C₃₀ fatty alcohol benzoates, and mixtures thereof.

Preferably the organic compounds are selected from compounds which are liquid at a temperature of 25° C. and under 10⁵ Pa (760 mmHg).

The compositions in accordance with the invention may further comprise at least one agent which is commonly used in cosmetology, selected, for example, from reducing agents, fats, plasticizers, softeners, antifoams, moisturizers, pigments, clays, mineral fillers, UV filters, mineral colloids, peptizers, solubilizers, perfumes, preservatives, anionic, nonionic or amphoteric surfactants, fixative or non-fixative polymers, polyols, proteins, vitamins, direct dyes or oxidation dyes, pearlizers, propellants, and organic or inorganic thickeners such as benzylidenesorbitol and N-acylamino acids.

These agents may be optionally encapsulated. The capsule may be of polycyanoacrylate type.

The propellant is used in particular for the preparation of aerosol compositions. It generally comprises compressed and/or liquefied gases which are well known in the art. Preference is given to using air, carbon dioxide, compressed nitrogen or else a gas such as dimethyl ether, non-halogenated or halogenated—fluorinated, for example—hydrocarbons, or a mixture thereof.

The composition is used on keratin materials, and more particularly on keratin fibres such as the hair, preferably in the presence of a nucleophile, for their cosmetic treatment.

The method of cosmetic treatment according to the invention comprises applying a composition as defined above to keratin materials, and more particularly to keratin fibres such as the hair, in the presence of a nucleophile as defined below.

The nucleophiles capable of initiating the anionic polymerization are systems known per se which are capable of generating a carbanion on contact with an electrophilic monomer. By carbanion is meant the chemical species defined in “Advanced Organic Chemistry”, Third Edition, Jerry March, page 141.

The nucleophiles may be composed of a molecular compound, an oligomer, a dendrimer or a polymer which possesses nucleophilic functions. Without limitation, nucleophilic functions which may be mentioned include the following functions: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph represents the phenyl group, Ar represents an aryl group and R represents a C₁-C₁₀ alkyl group.

Preferably, the nucleophiles are hydroxyl ions, such as those present in water. This water may be provided by prior wetting of the keratin materials.

It is also possible, in order to modify the reaction kinetics, to wet the keratin materials, and especially the keratin fibres such as the hair, beforehand by means of an aqueous solution whose pH has been adjusted using a base, an acid or an acid/base mixture. The acid and/or the base may be organic or inorganic.

Preferably, the composition containing the electrophilic monomer does not contain any nucleophile.

Still more preferably, the nucleophile is contained in a second composition, used just before or after the composition containing the electrophilic monomer

It is also possible to modify the anionic polymerization kinetics by pre-impregnating the keratin materials, and especially the keratin fibres such as the hair, using a nucleophile other than water. The nucleophile may be used pure, in solution or in the form of an emulsion, or may be encapsulated.

In order to modify the anionic polymerization kinetics it is also possible to enhance the nucleophilicity of the keratin materials, and particularly of the keratin fibres such as the hair, by chemically converting the keratin material.

By way of example of chemical conversion, mention may be made of the reduction of the disulphide bridges, of which the keratin is partly composed, to thiols, prior to application of the composition of the invention. In a non-exhaustive listing, mention may be made, as reductants of the disulphide bridges of which the keratin is partly composed, of the following compounds:
anhydrous sodium thiosulphate,
powdered sodium metabisulphite,
thiourea,
ammonium sulphite,
thioglycolic acid,
thiolactic acid,
ammonium thiolactate,
glycerol monothioglycolate,
ammonium thioglycolate,
thioglycerol,
2,5-dihydroxybenzoic acid,
diammonium dithioglycolate,
strontium thioglycolate,
calcium thioglycolate,
zinc formaldehyde-sulphoxylate,
isooctyl thioglycolate,
di-cysteine,
monoethanolamine thioglycolate.

In order to modify the anionic polymerization kinetics, and more specifically to reduce the polymerization rate of the monomers of the invention, it is possible to enhance the viscosity of the composition. To accomplish this it is possible to add to the composition of the invention one or more polymers which exhibit no reactivity with the monomers in accordance with the invention. In this context mention may be made, non-exhaustively, of poly(methyl methacrylate) (PMMA) or else of cyanoacrylate-based copolymers as are described in patent U.S. Pat. No. 6,224,622.

In order to improve, among other things, the adhesion of the poly(cyanoacrylate) formed in situ, it is possible to pretreat the fibre with any types of polymers, or to carry out a hair treatment before applying the composition of the invention, such as a direct dyeing or oxidation dyeing, a permanent waving operation or else a straightening operation.

Application of the compositions as described above may be followed or not by rinsing.

The compositions may be in the form of a lotion, spray or foam and may be applied as a shampoo or conditioner.

Another subject of the invention consists of a kit comprising a first composition containing at least one electrophilic monomer as defined above and optionally at least one free-radical and/or anionic polymerization inhibitor as defined above, and a second composition comprising in a cosmetically acceptable medium at least one non-polymeric organic salt having a melting point of less than 60° C. as defined above.

The invention further provides a method of cosmetic treatment of keratin materials, comprising at least two steps, one step comprising applying the non-polymeric organic salt having a melting point of less than 60° C. as defined above, and another step comprising applying at least one electrophilic monomer as defined above, the order of the steps being arbitrary.

One particular embodiment of the invention consists in applying the non-reductant organic acid before applying at least one electrophilic monomer.

The examples which follow are given by way of illustration of the present invention.

In the examples below, all amounts are indicated in per cent by weight of active substance relative to the total weight of the composition, unless indicated otherwise.

### EXAMPLES

### Examples of a Rinsed Composition

A shampoo according to the invention (Example 1) and three comparative shampoos (Comparative Examples 1, 2 and 3) were prepared from the following ingredients:

| | | Comp. | Comp. | Comp. |
|---|---|---|---|---|
| | Ex. 1 | Ex. 1 | Ex. 2 | Ex. 3 |
| Sodium laureth sulphate | 30% | 30% | 30% | 30% |
| Cocobetaine | 4% | 4% | 4% | 4% |
| Cocamide | 2% | 2% | 2% | 2% |
| monoisopropanolamine | | | | |
| 1-Methyl-3-hexylimidazolium | 2% | — | 1.5% | — |
| tetrafluoroborate | | | | |
| n-Octyl-2-cyanoacrylate | 1.5% | — | — | 5% |
| Polydimethylsiloxane | — | 1.5% | — | — |
| (250,000 cSt) | | | | |
| Sodium cetearyl sulphate | 0.8% | 0.8% | 0.8% | 0.8% |
| POLYQUATERNIUM-10 | 0.4% | 0.4% | 0.4% | 0.4% |
| CARBOMER 980 | 0.2% | 0.2% | 0.2% | 0.2% |
| Propylene glycol | 0.1% | 0.1% | 0.1% | 0.1% |
| Preservative | qs | qs | qs | qs |
| Perfume | qs | qs | qs | qs |
| Water qs | 100 | 100 | 100 | 100 |

### Application Method 1

2 g of each of the compositions of Example 1 and Comparative Examples 1 to 3 are applied to tresses consisting of 2.7 g of sensitized hair. After two minutes of contact between the compositions and the tresses of hair, the latter are rinsed and then dried for an hour at ambient temperature (approximately 20-25° C.).

### Application Method 2

Tresses consisting of 2.7 g of sensitized hair are wetted using 1 ml of water per tress. 2 g of each of the compositions of Example 1 and Comparative Examples 1 to 3 are applied to these wetted tresses. After two minutes of contact between the compositions and the tresses of hair, the latter are rinsed and then dried for an hour at ambient temperature (approximately 20-25° C.).

For each tress, the feel and the sheen of the hair are evaluated by a panel of 10 individuals. An untreated tress of the same kind is used as reference.

The tactile and visual evaluation of the various tresses of hair is repeated with the same procedure after 5 successive applications of shampoos sold under the name DOP camomile.

The results of sensorial evaluation are indicated in the following tables:

| | | | | |
|---|---|---|---|---|
| Type of | | | | |
| treatment | Ex. 1 | Comp. Ex. 1 | Comp. Ex. 2 | Comp. Ex. 3 |

Application method 1

| | | | | |
|---|---|---|---|---|
| After | softness 3 | softness 2 | softness 2 | softness 3 |
| application | sheen 4 | sheen 1 | sheen 4 | sheen 1 |
| After 5 | softness 2 | softness 0 | softness 0 | softness 2 |
| shampooings | sheen 3 | sheen 0 | sheen 1 | sheen 1 |

Application method 2

| | | | | |
|---|---|---|---|---|
| After | softness 4 | softness 2 | softness 2 | softness 4 |
| application | sheen 4 | sheen 1 | sheen 4 | sheen 1 |
| After 5 | softness 3 | softness 0 | softness 0 | softness 3 |
| shampooings | sheen 3 | sheen 0 | sheen 1 | sheen 1 |

The notation is as follows:
0 = equivalent to the untreated tress;
5 = greatly superior to the untreated tress.

The softness and sheen provided by the composition according to the invention are superior to or similar to the compositions of the comparative examples just after the composition has been applied.

After 5 shampooings, the imparting of softness and sheen is retained more effectively when the tresses are treated with the composition according to the invention of Example 1.

### Examples of an Unrinsed Composition

A sheen cream according to the invention (Example 2) and three comparative sheen creams (Comparative Examples 4, 5 and 6) were prepared from the following ingredients:

| | | Comp. | Comp. | Comp. |
|---|---|---|---|---|
| | Ex. 2 | Ex. 4 | Ex. 5 | Ex. 6 |
| 1-Methyl-3-hexylimidazolium | 1.5% | — | 1.5% | — |
| tetrafluoroborate | | | | |
| n-Octyl 2-cyanoacrylate | 5% | — | — | 5% |
| Cyclopentasiloxane | 10% | 10% | 10% | 10% |
| Cyclopentasiloxane | 0.5% | 0.5% | 0.5% | 0.5% |
| dimethicone copolyol | | | | |
| Propylene glycol | 2.5% | 2.5% | 2.5% | 2.5% |
| Preservative | qs | qs | qs | qs |
| Perfume | qs | qs | qs | qs |
| Water qs | 100 | 100 | 100 | 100 |

### Application Method 1

2 g of each of the compositions of Example 2 and the comparative examples 4 to 6 are applied to tresses consisting of 2.7 g of sensitized hair. Following application, the tresses of hair are dried under a hood at 70° C. for 30 minutes.

### Application Method 2

Tresses consisting of 2.7 g of sensitized hair are wetted with 1 ml of water per tress. 2 g of each of the compositions of Example 2 and Comparative Examples 4 to 6 are applied to these wetted tresses. Following application, the tresses of hair are dried under a hood at 70° C. for 30 minutes.

For each tress, the feel and the sheen of the hair are evaluated by a panel of 10 individuals. An untreated tress of the same kind is used as reference.

The tactile and visual evaluation of the various tresses of hair is repeated with the same procedure, following 5 successive applications of shampoos sold under the name DOP camomile.

The results of sensorial evaluation are indicated in the tables below:

| | | | | |
|---|---|---|---|---|
| Type of | | | | |
| treatment | Ex. 2 | Comp. Ex. 4 | Comp. Ex. 5 | Comp. Ex. 6 |

Application method 1

| | | | | |
|---|---|---|---|---|
| After | softness 4 | softness 3 | softness 3 | softness 4 |
| application | sheen 5 | sheen 3 | sheen 5 | sheen 2 |
| After 5 | softness 3 | softness 0 | softness 0 | softness 3 |
| shampooings | sheen 4 | sheen 0 | sheen 1 | sheen 1 |

Application method 2

| | | | | |
|---|---|---|---|---|
| After | softness 5 | softness 3 | softness 3 | softness 5 |
| application | sheen 5 | sheen 3 | sheen 5 | sheen 2 |
| After 5 | softness 4 | softness 0 | softness 0 | softness 4 |
| shampooings | sheen 4 | sheen 0 | sheen 1 | sheen 1 |

The notation is as follows:
0 = equivalent to the untreated tress;
5 = greatly superior to the untreated tress.

The softness and sheen provided by the composition according to the invention are superior to or similar to the compositions of the comparative examples just after the composition has been applied.

After 5 shampooings, the imparting of softness and sheen is retained more effectively when the tresses are treated with the composition according to the invention of Example 2.

### Other Examples of Unrinsed Compositions

### Example 3

| | Ex. 3 | Comparative |
|---|---|---|
| 1-Methyl-3-hexylimidazolium | 1.5% | — |
| tetrafluoroborate | | |
| Methylheptyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*sold by the Chemence company

### Example 4

| | Ex. 4 | Comparative |
|---|---|---|
| 1-Methyl-3-hexylimidazolium | 1.5% | — |
| tetrafluoroborate | | |
| Methoxyethyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*MO 460 sold by the Tong Shen company

### Example 5

| | Ex. 5 | Comparative |
|---|---|---|
| 1-Methyl-3-hexylimidazolium | 1.5% | — |
| tetrafluoroborate | | |
| Ethoxyethyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*EO-460 sold by the Tong Shen company

### Example 6

| | Ex. 6 | Comparative |
|---|---|---|
| 1-Methyl-3-hexylimidazolium | 1.5% | — |
| tetrafluoroborate | | |
| Butyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*B-60 sold by the Tong Shen company

### Example 7

| | Ex. 7 | Comparative |
|---|---|---|
| 1-Methyl-3-hexylimidazolium | 1.5% | — |
| tetrafluoroborate | | |
| Ethyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*Cyanolit 202 sold by the Loctite company

### Example 8

| | Ex. 8 | Comparative |
|---|---|---|
| 1-Methyl-3-hexylimidazolium | 1.5% | — |
| tetrafluoroborate | | |
| Ethylhexyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*O-60 sold by the Tong Shen company

### Example 9

| | Ex. 9 | Comparative |
|---|---|---|
| 1-Methyl-3-hexylimidazolium | 1.5% | — |
| tetrafluoroborate | | |
| n-Octyl cyanoacrylate* | 5% | 5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*sold by the Chemence company

### Example 10

| | Ex. 10 | Comparative |
|---|---|---|
| 1-Methyl-3-hexylimidazolium | 1.5% | — |
| tetrafluoroborate | | |
| Methylheptyl cyanoacrylate* | 4.5% | 4.5% |
| Ethylhexyl cyanoacrylate** | 0.5% | 0.5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*sold by the Chemence company
**O-60 sold by the Tong Shen company

### Example 11

| | Ex. 11 | Comparative |
|---|---|---|
| 1-Methyl-3-hexylimidazolium | 1.5% | — |
| tetrafluoroborate | | |
| Methylheptyl cyanoacrylate* | 3.5% | 3.5% |
| Butyl cyanoacrylate** | 1.5% | 1.5% |
| Cyclopentasiloxane | 10% | 10% |
| Cyclopentasiloxane dimethicone | 0.5% | 0.5% |
| copolyol | | |
| Propylene glycol | 2.5% | 2.5% |
| Preservative | qs | qs |
| Perfume | qs | qs |
| Water qs | 100 | 100 |

*sold by the Chemence company
**B-60 sold by the Tong Shen company

1. Composition comprising in a cosmetically acceptable medium at least one electrophilic monomer and at least one non-polymeric organic salt having a melting point of less than 60° C.

2-43. (canceled)
